(19) United States
(12) Patent Application Publication
(10) Pub. No.: US 2010/0003666 A1
(43) Pub. Date: Jan. 7, 2010

(54) Microfluidic Methods for Diagnostics and Cellular Analysis

(75) Inventors: Luke P. Lee, Orinda, CA (US); Dino Di Carlo, Boston, MA (US); Joshua Tanner Nevill, El Cerrito, CA (US)

Correspondence Address: BUCHANAN, INGERSOLL & ROONEY LLP, P.O. BOX 1404, ALEXANDRIA, VA, 22313-1404, US

(73) Assignee: THE REGENTS OF THE UNIVERSITY OF CALIFORNIA, OAKLAND, CA (US)

(21) Appl. No.: 11/990,130

(22) PCT Filed: Aug. 18, 2006
(86) PCT No.: PCT/US06/32355

§ 371 Date: Aug. 28, 2009

Related U.S. Application Data
(60) Provisional application No. 60/709,574, filed on Aug. 19, 2005.
(60) Provisional application No. 60/709,593, filed on Aug. 19, 2005.

(51) Int. Cl. C12Q 1/70; C12Q 1/02; C12M 1/34

(52) U.S. Cl. 435/5; 435/29; 435/287.1

(57) ABSTRACT

Methods for detection of molecular recognition and analysis of cells are provided. Both optical and non-optical methods are presented. Methods utilize capture of particles in semi-permeable structures. Specific microfluidic system architectures for conducting biomolecule and cell assays are described.

### CROSS REFERENCE TO RELATED APPLICATIONS

This application claims priority to U.S. Provisional Application Ser. Nos. 60/709,574 and 60/709,593, both filed Aug. 19, 2005, the disclosures of which are incorporated herein by reference.

### FIELD OF THE INVENTION

The invention relates to the fields of analytical and bio-analytical methods and more specifically to microfluidic analysis systems, lab-on-a-chip systems, and micro total analysis systems.

### BACKGROUND

Analysis of molecular binding and cell behavior are important for disease diagnostics, biomedical research, and drug discovery. Accordingly, many analysis methods have been developed. However, improved methods are needed that facilitate rapid detection.

### SUMMARY

The invention provides a method for performing large scale single cell and analyte analysis which has significant advantages over flow cytometry and laser scanning cytometry (LSC). Trapping arrays in a microfluidic format allow for high density analysis and ease image processing. Moreover, on-chip sample preparation using the invention saves time and reagents. Additionally, time-dependent phenomena of a large number of single cells over different time scales are capable of being characterized using this device. The methods of the invention are well-suited to high throughput quantitative biology where dynamics of single cells can be observed to provide rapid medical screening.

The invention provides a microfluidic device useful as a biological trapping system or micro-affinity column. The device comprises a chamber having an inlet; an outlet; and at least one substrate, wherein the substrate is disposed between and in fluid communication with the inlet and the outlet; a cover, wherein the cover and the at least one substrate define an internal flow space, the flow space having a height, and a plurality of weir-traps, wherein the plurality of weir-traps are disposed on the substrate and extend into the internal flow space, the plurality of weir-traps having a height less than the height of the flow space. The fluidic device can further comprise additional elements including, but not limited to, a buffer reservoir fluidly connected to the chamber; a means for measuring fluid flow through the chamber; one or more electrodes for electrically measuring an analyte or biological agent located with the chamber; a plurality of beads (e.g., affinity-based beads) located between at least two weir-traps of the plurality of weir-traps within the chamber, the bead may be functionalized. The device may also include one or more pumps and/or valves.

The invention also provides a method for the detection of a target analyte or biological agent in a fluid sample. The method includes contacting the sample with a microfluidic device of the invention and detecting a change in the devices electrical or flow properties or optics.

The details of one or more embodiments of the disclosure are set forth in the accompanying drawings and the description below. Other features, objects, and advantages will be apparent from the description and drawings, and from the claims.

### BRIEF DESCRIPTION OF DRAWINGS

FIG. 1 shows a typical Sandwich Assay. A fluorescent label is required to determine if the binding event was successful.

FIG. 2 shows concentration dependent behavior for traditional agglutination assays. A phase diagram schematically showing the aggregate type for various ratios of antigen/molecule of interest to microparticle/cell concentration is depicted. There is a small region where large aggregates will form, but a large amount of regions where no large aggregates will form even for very high concentrations of molecule of interest.

FIG. 3 depicts macroscale methodology to make concentration independent agglutination assays. A multiple step process must be performed on the macroscale, including centrifugation, and resuspension. This type of process usually fails since these are high shear processes that lead to shearing apart of the formed aggregates.

FIG. 4A-C depicts a microfluidic single cell isolation array of the invention. A schematic diagram of an embodiment of the microfluidic device is shown. Branching delivery channels insure a substantially equal distribution of flow with cells and reagents to each trapping array. Only one inlet and outlet are depicted, however, additional ports can be used. The inset shows more details of the device in a micrograph of a pair of trapping arrays.

FIG. 5A-C depict a high density single cell isolation device and method of the invention. (a-b) A schematic diagram is shown depicting a mechanism of cell trapping using flow through arrayed suspended obstacles. Two-layer (40 μm and 2 μm) cup-shaped PDMS trapping sites allow a fraction of fluid streamlines to enter the traps. After a cell is trapped and partially occludes the 2 μm open region, the fraction of streamlines through the barred trap decreases, leading to the self-sealing quality of the traps and a high quantity of single cell isolates. Drawing is not to scale. (c) A phase contrast image of an array of single trapped cells is shown. The scale bar is 30 μm.

FIG. 6A-E shows statistics of single-cell isolation. (a-d) Phase contrast micrographs of cell trapping in varying geometry cell isolation traps are shown. From a-d trap depth varied as 10 μm, 15 μm, 30 μm and 60 μm. The number of cells trapped scales with the trap size, with more trapping of single cells observed as the trap size decreases. (e) The distribution of trapped cells for the geometry shown in (a) is plotted along with a Poisson distribution for the same average value. If the probability of trapping was independent of the amount of previously trapped cells one would expect a Poisson distribution. In this case an enhancement of single cell containing traps and a reduction of zero and greater than two cell containing traps is observed above the random process. Here data from four separate loadings of 100 μL of cell solution containing approximately 3×10⁶ cells ml⁻¹ was flowed through the device before data was collected.

FIG. 7 shows a procedure for concentration independent agglutination assays. Aggregates are built up over many steps on semi-permeable structures similar to those used for cell trapping. Valves can be used to switch between the microparticle/cell phase and the molecule to be detected. Aggregates do not break up in this situation as compared to centrifugation and resuspension. After completely obstructing the channel, beads pile up and are observable by the naked eye as a positive signal of molecule presence. Additionally electrical measurements could be performed directly to observe the obstruction of the channel.

FIG. 8A-G show a schematic overview of a device, assay methodology, and measurement concept of the invention. (A) Layout of the device. (B) Functionalized beads in solution are loaded into the channel, and beads are packed into a designated region in between two ‘dams.’ (C) Buffer is pushed through the bead pack, and a resistance measurement is taken with two electrodes. (D) Sample is passed through the bead pack. (E) Sample is washed out with buffer, and resistance measurement is recorded. If molecules specific to the functionalized beads are present, they coat the beads which increases the resistance through the bead pack. (F) Cross section of the bead pack region as shown in part C. (G) Cross section of bead pack as shown in part E.

FIG. 9A-C shows calculations for trapping and detection using methods and devices of the invention. (A) The unit cell for the nanocavity calculations is shown. Packed microparticles lead to z dependent areas and perimeters that repeat with the unit cell. Perimeters allow calculation of obstructed area with binding of biomolecules. (B) The z dependence of area and perimeter through a nanocavity system created by a 3 μm radius microparticle pack is plotted. Regions of the graph where area is minimized and perimeter is maximized lead to the highest resistance increases upon binding. (C) Resistance ratio upon biomolecule binding as a function of the bead pack radius is plotted for various sized biomolecules.

FIG. 10A-C depicts a flow and an assay methodology of the invention. (A) Cartoon of the immunochromatographic sandwich assay. (B) Sample can travel through the void spaces in between the packed beads. (C) If gold nanoparticles are conjugated to the surfaces of the beads, light is scattering to an extent that is visible to the naked eye.

FIG. 11 depicts a large scale single cell trapping device of the invention. Top down drawing of large scale trapping array with a high trapping density of 25,000 traps per square cm is shown. Branching inlets and outlets allow more uniform flow to every area of the trapping array.

FIG. 12A-B depict a single cell isolation arrays for cell filtering. (a) A 3D drawing of the mechanism of cell trapping is shown. (b) Two-layer (40 μm and 10 μm) cup-shaped PDMS trapping sites suspended from the glass substrate allow a fraction of fluid streamlines to enter the traps. After a cell is trapped and partially occludes the 10 μm open region, the fraction of streamlines through the barred trap decreases, leading to a high quantity of single cell isolates. Drawing is not to scale. Leukocytes, RBCs and platelets having a smaller diameter (6-12 μm) compared to >20 microns for circulating tumor cells (CTCs) can pass through the trapping structures. In this way CTCs are isolated and concentrated.

FIG. 13A-C shows an embodiment of a single cell trapping array of the invention. (A) A photograph of the cell trapping device is shown demonstrating the branching architecture and trapping chambers with arrays of traps. The scale bar is 500 μm. Cell and media flow enters from the left and enters the individual trapping chambers where it is distributed amongst the individual traps. (B) A diagram of the device and mechanism of trapping is presented. Traps are molded in PDMS and bonded to a glass substrate. Trap size biases trapping to predominantly one or two cells. The diagram is flipped from the actual device function for clarity; a functioning device is operated with the glass substrate facing down towards the earth. An inset shows the geometry of an individual trap. The device is not drawn to scale. (C) A high resolution brightfield micrograph of the trapping array with trapped cells is shown. In most cases cells rest at the identical potential minimum of the trap, while in some cases two cells are trapped in an identical manner amongst traps. A magnification shows the details of the trapped cell. Trapping is a gentle process and no cell deformation is observed for routinely applied pressures.

FIG. 14A-B shows modeling shear stress. Velocity magnitude and shear stress magnitude is plotted for a 3D model of the trapping structure with a trapped spherical cell. Velocity magnitude is plotted for a z distance 20 μm from the substrate, while shear stress magnitude is plotted for the boundary surface of the microchannel and trapped cell. (A) Velocity magnitude is plotted showing a region of reduced velocity within the trapping structure. The scale goes from a maximum of 50 μm s⁻¹ to a minimum of 0 μm s⁻¹. (B) Shear stress magnitude is plotted on the lower boundary of the device. Here the scale extends from 0 to 0.12 dyn cm⁻² in the main graph. An inset shows a close-up of the trapping region with a new scale extending from 0 to 0.025 dyn cm⁻². Notice the reduced shear stress within the trapping structure. Scale bars are 25 μm.

FIG. 15A-C shows an arrayed single cell culture. Micrograph images of cells cultured within the microfluidic arrays are shown. Cells were cultured under continuous perfusion of media+10% FBS with an average velocity (25 μm s⁻¹) for over 24 hours. Pictures are shown at times (A) 0 hrs, (B) 12 hrs, and (C) 24 hrs. The arrows indicate cells that undergo cell division within this time period. Scale bar is 50 μm.

FIG. 16 shows uniform cell behavior in an array. Characteristics of growth for single trapped cells are shown. Frames from a movie of cell growth in the array are shown demonstrating both cell division (first three rows) and morphologies indicative of cell adhesion (rows 4 through 6). Notice the uniformity in morphology observed amongst adherent and amongst dividing cells. The hours after seeding are shown underneath each image. After division daughter cells remained within the trapping region.

FIG. 17A-B shows cell behavior in trapping structures and the control substrate. (A) Cell adhesion, division, and death are reported every hour for individual cells in the single cell array. (B) The same characteristics are plotted for culture on a control glass slide without perfusion.

FIG. 18A-B shows morphology in trapping structures and control substrate. HeLa cell morphology is shown after 24 hour growth on a glass substrate without perfusion (A) and after 24 hours of perfusion in the trapping array (B). Notice the similar adherent morphology. Some differences are observed due to attachment of cells to the PDMS structures in (B). Scale bars are 25 μm.

### DETAILED DESCRIPTION

Unless defined otherwise, all technical and scientific terms used herein have the same meaning as is commonly understood by one of skill in the art to which this invention belongs. All publications and patents referred to herein are incorporated by reference.

As used herein and in the appended claims, the singular forms “a,” “and,” and “the” include plural referents unless the context clearly dictates otherwise. Thus, for example, reference to “an analyte” includes a plurality of such analytes and reference to “the valve” includes reference to one or more valves known to those skilled in the art, and so forth.

For biomolecule recognition the most common methods employ fluorescence or chemiluminescent detection. In this scheme a recognition element with a label element is bound to a molecule of interest and unbound recognition element is removed by a method of washing (Marquette et al., Biosensors & Bioelectronics 2006, 21, 1424-1433). Thus a selective signal is produced only when binding occurs.. In most cases the recognition element is immobilized on a surface. These techniques usually require a complicated optical system with a light source, filter, and detector before a signal of binding is determined. In most cases a large microscope or microplate reader will be used for this purpose. A common commercial implementation of this technique is the ELISA (Engvall et al., Journal of Immunology 1972, 109, 129). For portable and cheap diagnostic systems purely electrical detection without optical transduction is desired. A variety of chip-based microfluidic devices have also been developed as diagnostic systems using optical or electrochemical transduction of biomolecule recognition (Yager et al., Nature 2006, 442, 412-418; Warsinke et al., Journal of Analytical Chemistry 2000, 366, 622-634). Here the system integration of optical components or repeatability of electrochemical measurements can be limiting.

Microparticles are often used as a stationary site for recognition element immobilization in macroscale (Yingyongnarongkul et al., Combinatorial Chemistry & High Throughput Screening 2003, 6, 577-587) and microfluidic assays (Verpoorte, E., Lab on a Chip 2003, 3, 60n-68n). This is because particles with different elements can be interchanged easily to conduct different assays and the surface area for immobilization is large leading to large optical signals that increase with decreasing particle size. However, microparticle or bead-based bioassays have been limited to fluorescent or colorimetric detection of binding.

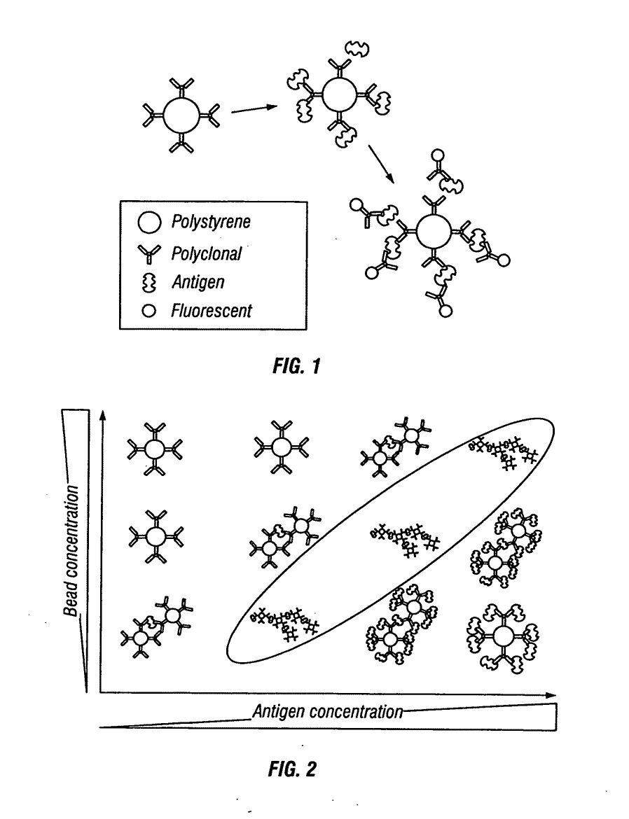

One of the most typical bead based assay uses a ‘sandwich assay’ (see FIG. 1), where an antibody is attached to the bead surface. An antigen, if it is specific to the antibody, will then bind to the beads. Then, a second antibody that is tagged with a fluorescent molecule is attached to the antigen, forming a ‘sandwich’. This method works well, but a second labeling step is required, and the fluorescence must be monitored with an optical system, a light source, and a photodetector.

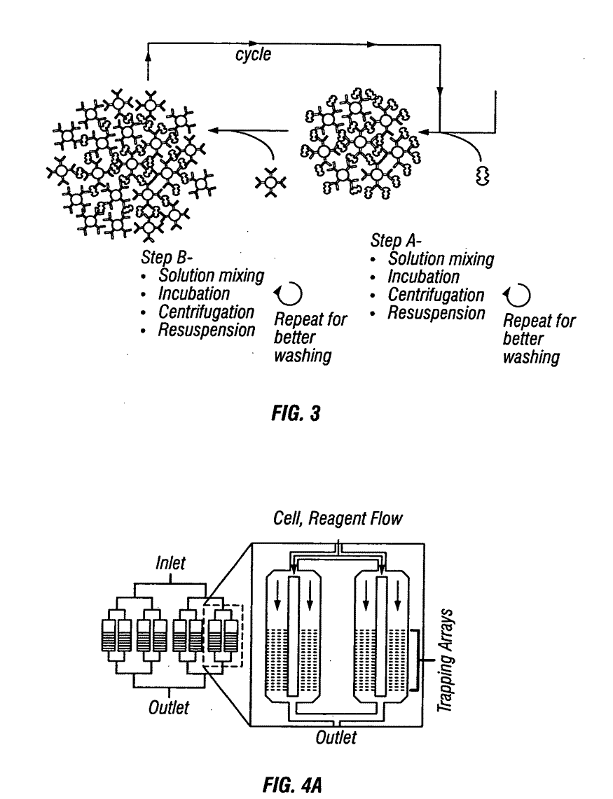

Another method for detecting biomolecules and their interactions is the bead agglutination assay (Coombs et al., British Journal of Experimental Pathology 1945, 26, 255-266; Reis et al., Transfusion 1993, 33, 639-643). In this assay the aggregation of multiple beads or cells into clumps mediated by some biomolecular recognition event is usually detected as a change in the optical properties of a solution containing the suspended beads/nanoparticles. In this type of system the aggregation is very dependent on the ratio of microparticles/nanoparticles to molecule of interest (FIG. 2) and so will only lead to a positive detection if the concentration is within an optimum range. Macroscale methods can be utilized to increase this range, but require centrifugation and resuspension of aggregate (FIG. 3). These manual operations are not easily performed in a portable system, and are expensive and time consuming.

Flow cytometry (FC) and laser scanning cytometry (LSC) are some of the most widely used techniques for single cell analysis. Well characterized distributions of cellular behavior are often observed using flow cytometry. Briefly, this technique involves the hydrodynamic isolation of individual cells that have been previously labeled using fluorescent dyes that reveal information about the quantity of biomolecules of interest within that cell. A light source, filtering mechanism, and detector are present to observe these signals of individual cells. The technique is a very high throughput serial process, where in most cases cells are discarded after analysis.

Flow cytometry has been the most successfully used technique for single cell analysis because of the massive throughput; however it has been limited in most cases to characterizing fluorescent signals (GFP-fusion proteins, immunofluorescence, and fluorogenic substrates to intracellular enzymes)(Fayet al., Biochemistry 1991, 30, 5066-5075; Nolan et al., Nature Biotechnology 1998, 16, 633-638; and Krutzik et al., Nature Methods 2006, 3, 361-368). Additionally, it does not address important time dependent measurements of the same individual cell, or spatial localization of fluorescence within a cell. Cells analyzed using this method are usually grown in a flask or dish before analysis, and so uniformity of environment is limited to that of the flask or dish. Notably, cell-cell contact is not controllable, and diffusible secretions are maintained in the culture environment.

Laser scanning cytometry (LSC), a technique where dyes on surface immobilized cells are excited by a scanning laser, and can be repeatedly interrogated in time is an alternative technique that has been employed (Griffin et al., Febs Letters 2003, 546, 233-236; Bedner et al., Cytometry 1998, 33, 1-9). Here, an advantage over FC, time dependent information can be obtained in individual cells, and adherent cells can be maintained in the primary site of culture during analysis. However, LSC sacrifices throughput as only a limited region of a plate can be scanned. Additionally, time and throughput has somewhat of a tradeoff, as scanning more cells will lead to an increased time between measurements for individual cells. For LSC introduction of reagents is done by pipette and only slow time dependent changes after solution exchange are meaningful. This is particularly due to uneven introduction of solution over the whole slide or plate, and the serial process of laser scanning. As in FC, the cells are maintained on a slide or dish, and the environment is not well controlled.

In order to address the aspects of environmental control, fast timescale measurements, image processing, and secreted biomolecule isolation, several methods of single cell isolation have been developed. A number of microfluidic techniques have been reported to allow optical interrogation of individual cells integrated with fast exchange of reagents.

In general, microfluidic techniques employ microfabrication for the miniaturization of fluid channels and conduits. Systems of channels and structures are created that allow dynamic control of reagents and cells through fluid perfusion, and pressure gradients. Most techniques require complicated operation or fabrication to isolate individual cells.

The invention describes the use of semi-permeable obstacles (referred to herein as “weir-traps”) to passively create uniform arrays of individually trapped cells or analytes within a microfluidic platform. In some aspects, the invention provides such a platform that does not required optical feedback. In this platform, the microenvironment is well controlled for individual cells and analytes, including contact and diffusible stimuli, by isolation and perfusion, respectively. The weir-trap structure of the invention has features that allow passive trapping of single cells and analytes in arrays in less than 30 seconds. Changing trap geometry also allows engineering of the number of cell-cell (or binding partners, e.g., antigen-antibody) contacts by trapping groups of cells or analytes in proximity. Although throughput is reduced when compared with that of FC, microfluidic integration allows fast timescale measurements of tens to hundreds of single cells in parallel.

As described further herein, the invention provides a method whereby a binding event between small molecules can be detected through simple and inexpensive pressure-based or impedance-based measurements as well as hybrid integration of low cost photodiodes light source and detectors. Thus, eliminating the need for expensive optics normally associated with bead-based assays. Such a device is useful as a portable point-of-care diagnostic device.

The invention provides a microfluidic tools to conduct simplified concentration independent aggregation for bioassays. Thus, eliminating the need for centrifugation and resuspension and increasing the sensitivity range for detecting aggregation. The invention devices, systems, and methods can be used in Immunoassays, Point-of-care diagnostics, DNA hybridization detection, Blood-typing, Single Cell Analysis as well as in cancer detection, minimal residual disease detection, high throughput screening of platelet activation in a variety of clinical conditions—for cardiovascular disease treatment, high throughput screening of pharmaceuticals modifying cell behavior, rare cell detection in blood, and in vitro toxicological screening, to name but a few utilities.

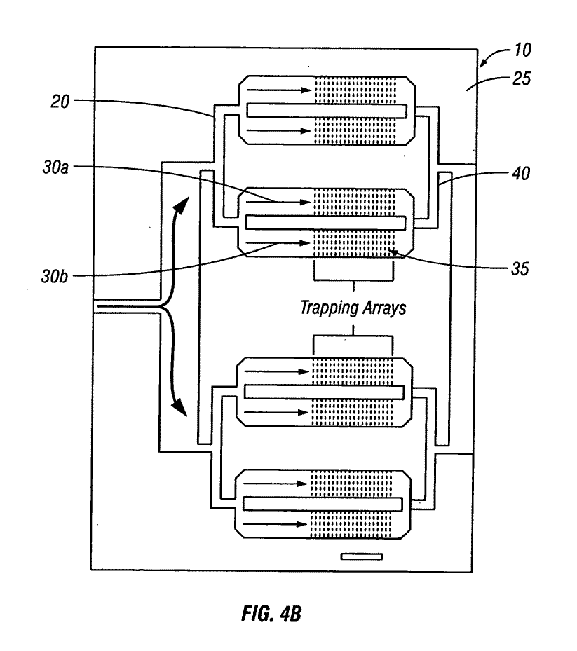

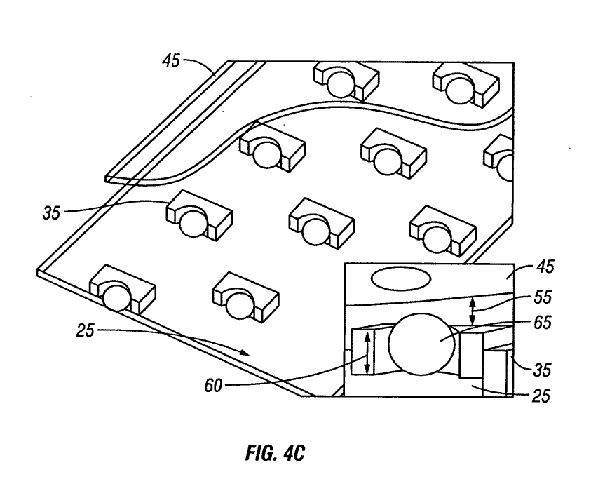

An exemplary fluidic device 10 of the invention is illustrated in FIG. 4B. The fluidic systems of the device 10 are disposed on a substrate 25. The substrate 25 can be any material useful for forming fluidic channels. A surface of the substrate and/or weir-trap may be modified to make it suitable for attachment of binding ligands (e.g., biological molecules). Substrates useful in the device include, but are not limited to, metal, glass, and plastic that may be used directly or may be modified with coatings (e.g., metals or polymers). The substrate can be a metal, glass or silicon surface. In a one embodiment, the substrate can be made from a wide variety of materials, including, but not limited to, silicon such as silicon wafers, silicon dioxide, silicon nitride, glass and fused silica, gallium arsenide, indium phosphide, aluminum, ceramics, polyimide, quartz, plastics, resins and polymers including polymethylmethacrylate, acrylics, polyethylene, polyethylene terepthalate, polycarbonate, polystyrene and other styrene copolymers, polypropylene, polytetrafluoroethylene, superalloys, zircaloy, steel, gold, silver, copper, tungsten, molybdeumn, tantalum, KOVAR, KEVLAR, KAPTON, MYLAR, brass, sapphire, and the like. High quality glasses such as high melting borosilicate or fused silicas may be used for their UV transmission properties when any of the sample manipulation steps require light based technologies. In addition, portions of the device may be coated with a variety of coatings as needed, to reduce non-specific binding, to allow the attachment of binding ligands, for biocompatibility, for flow resistance, and the like.

The fluidic device 10 comprises at least one inlet port 20 fluidly connected to at least one flow chamber 30 (depicted are flow chambers 30a and 30b). The flow chamber 30 comprises a plurality of trapping weir-traps 35. The weir-traps 35 may be located throughout the flow chamber 30, or may be located in a region proximal or distal to the inlet port 20. A plurality of weir-traps 35 can be referred to as an array. The flow chamber 30 is fluidly connected to at least one outlet port 40 typically at the chamber's distal end (although other outlet ports may be arranged anywhere within the chamber 30).

The microfluidic inlet port 20, chamber 30, outlet port 40 and any other fluid channels may be formed by any suitable micromachining technique into a suitable material, such as a silicon wafer. Ideally the material chosen should be capable of being sterilized and should not pose a biological threat to biological agent (e.g., cells, polypeptides and the like) that may be used in the fluidic device. The fluid flow regions of the device 10 are typically designed in a substrate 25 that is then sealed through a cover 45 (see, e.g., FIG. 4C). The cover 45 or substrate 25 may be formed of a transparent material. The transparent material allows convenient visual monitoring of cells or other biological material in the device. The cover may be attached (permanently or removably) by any number of means known in the art. For example, a bonding agent such as an epoxy or other glue can be used. In one aspect, the cover 45 is slidably mounted within the chamber 30 so that the cover 45 can be moved up or down within the chamber thereby increasing the volume of the chamber 30. Methods of sealing the cover in such an embodiment are known in the art.

Referring to FIG. 4C there is shown in further detail the flow chamber 30 comprising weir-traps 35 and cover 45 (see also FIG. 13B), also exemplified is biological agent 65 (e.g., cell or analyte). As described further herein, weir-traps 35 are located within chamber 30 to substantially, but not fully, reduce fluid flow with in chamber 30. Weir-traps 35 are designed to be of sufficient size to capture/trap cells or analytes within a fluid flowing through chamber 30. For example, where the distance between the substrate surface in fluid contact and the cover surface in fluid contact is 42 microns (see, e.g., FIG. 13B), the weir-trap extends into the fluid flow space of fluid chamber 30 a distance that would prevent a biological agent 65 from flowing over the weir-trap 35. In the example, depicted in FIG. 4C and FIG. 13B, wherein the distance between substrate surface and cover surface is 42 microns, the weir-trap 35 comprises a weir-trap height 60 that extends about 40 microns into the fluid flow space of the fluid chamber 30. Thus, approximately 2 microns remain as a reduced fluid flow space 55. In operation, the weir-trap 35 is of sufficient depth into the flow space of chamber 30 to inhibit passage of (“trap”) a biological agent 65 (e.g., a cell or analyte), while allowing fluid passage through reduced fluid flow space 55.

In one embodiment, weir-traps 35 are capable of being retracted in the fluid flow space to allow passage of clearing of the flow chamber 30 following analysis. Such methods include actuation of selanoids or other techniques. In one embodiment, the surface opposite the weir-trap is moved to a greater distance from the weir-trap thus increasing the reduced fluid flow space 55. For example, following measurement or analysis, the weir-traps are retracted into the substrate or the distance between the weir-trap and the opposing surface (e.g., the cover) is increased to allow fluid flow and passage of the biological agent out of the flow chamber.

Weir-trap 35 can be any shape which prevents passage of (“traps”) a biological agent 65 while allowing fluid flow in a reduced fluid flow space 55 associated with the weir-trap 35. For example, as depicted in the accompany figures weir-trap 35 has a concave shape.

Trapping array geometries are typically designed in a staggered fashion to optimize biological agent trapping as depicted in FIG. 4C. With the geometries and flows is possible to trap a plurality of biological agents in a trapping array.

In one aspect, the weir-trap 35 may comprise a binding agent useful for binding an agent and providing aggregation. In this aspect, a binding ligand can be permanently or removably immobilized on a weir-trap surface. If a target analyte is present in the sample, the binding ligand will capture the target analyte. The fluid sample, will typically comprise the target analyte and functionalized beads comprising binding agents.

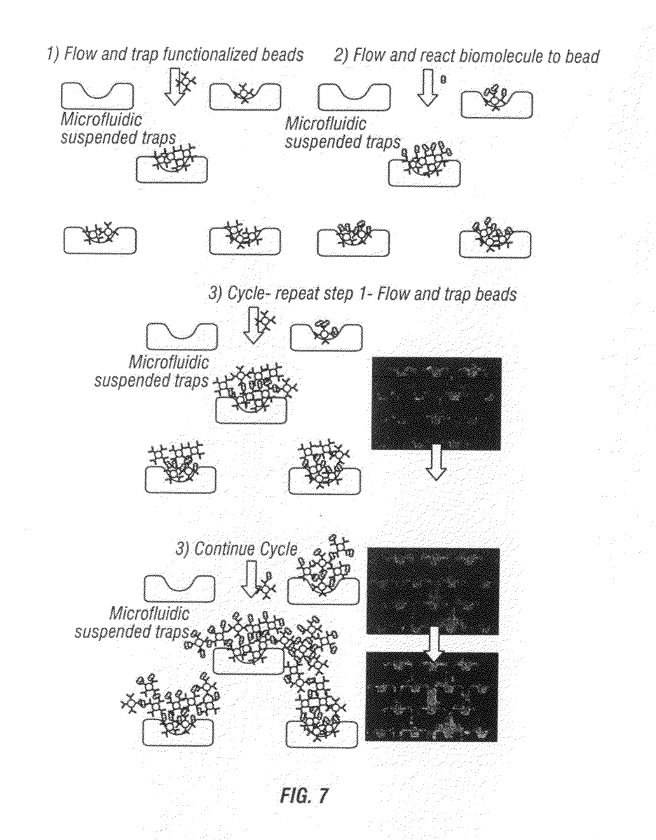

In another aspect, a first fluid comprising a functionalized bead comprising a binding agent is fluidly passed through the microfluidic device 10 followed by a fluid sample comprising a target agent (see, e.g., FIG. 7).

The process comprises, in one aspect, trapping of the target analyte from solution (e.g., immunospecific capture) followed by aggregation of additional target agents and binding agents. Trapping of aggregates will occur within the trapping array. The binding agents can be immobilized directly to weir-traps (where desired) by various chemistries and physical properties such as direct derivatization with biotin, and linkage with streptavidin or functionalized alkanethiols bound to gold pads.

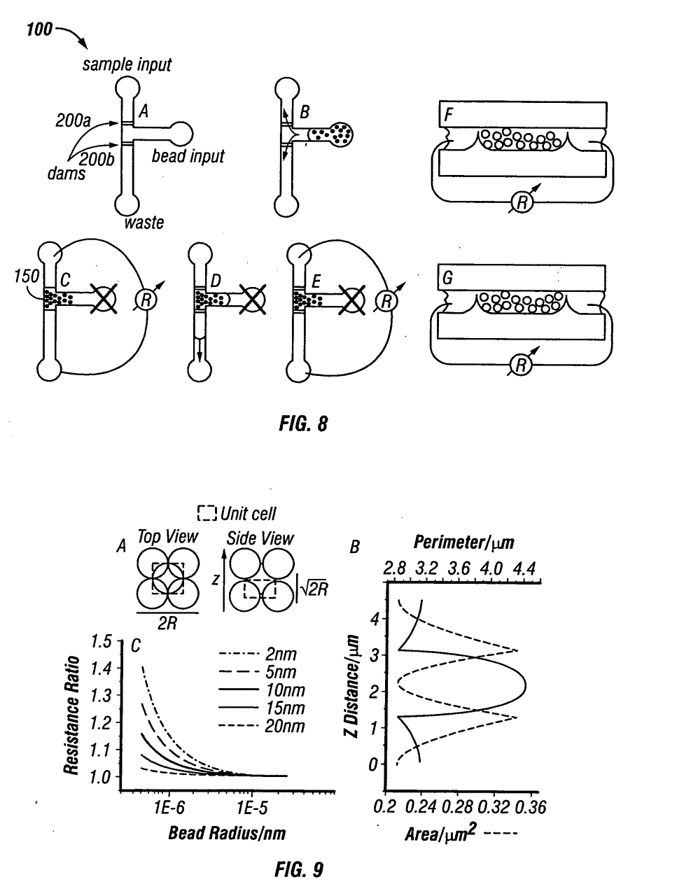

In another aspect of the invention a micro-affinity fluidic column is provided. Referring to FIG. 8A-C, the micro-affinity fluidic column 100 comprises a region of tightly-packed microspheres, or beads 150. The beads can be made of any material and the surfaces can be functionalized. Typically, the beads will be functionalized off chip, and then loaded into the micro-affinity fluid column 100. Two weir-traps (e.g., dams) 200a and 200b-in the fluidic column 100 will catch the beads in order to create the micro-affinity fluidic column 100. Once the beads are loaded, the device could be dried and then shipped and/or stored. FIG. 8C-G demonstrates the basic idea of how the micro-affinity fluidic column 100 is used. Sample is pushed through the bead pack 150 (see, e.g., FIG. 8D). If a target analyte or biological agent is present, it will specifically bind to the functionalized beads 150. The advantage of the column over other techniques is that the diffusion distance between the antibody and antigen is extremely small. This means that the assay time is minimized, and sensitivity is maximized. The entire sample is forced through the micro-affinity fluidic column 100, allowing for maximum antibody-antigen interaction. FIG. 8E depicts how the beads will be coated with antibody after the sample has been washed out. FIG. 8F and 8G represent cross-sections of the column region in (C) and (E), respectively. The micro-affinity fluidic column technique works in both glass and PDMS. Additionally, different types of beads are capable of being loaded into the same column by subsequently loading different bead solutions. One advantage of this technique is that control experiments could be performed simultaneously in the same channel. Different sized beads can also be packed (including nanoparticles) by simply introducing smaller and smaller beads into a fluidic channel of the micro-affinity fluidic column 100.

Methods are based on creating a nanocavity system with molecule binding sites, where the nanocavities decrease in dimension by an appreciable fraction upon binding of molecule of interest, but not non-specific molecules. Previously, it has been shown that beads coated with a protein will lead to an increased electrical resistance of a nanopore they are passing through, when compared to the uncoated bead. In that situation a time dependent dynamic measurement of resistance is required, and beads are pre-treated before analysis. However, in the invention, the measurement is made in a stationary nanocavity system which is directly treated with analyte (FIG. 8). Binding of analyte then decreases the cross-sectional area and increases the measured fluidic and electrical resistance across the nanocavity system. The nanocavity system can be formed by packing microparticles (beads) or in a stationary polymer phase. Because the functionalized beads can be prepacked in simple devices, there is great potential for use as disposable point-of-care diagnostic devices.

Detection of a trapped biological agent (e.g., a cell or polypeptide) can be performed in any number of methods (e.g., flow rate, flow resistance, electrical detection or optical detection). Electrical detection can be achieved through conductance, capacitance or charge based detection. Alternatively detection can be achieved optically, by a local optical stimulus and subsequent detection, e.g. through fluorescence.

Analysis instrumentation may be operably associated with each individual weir-trap or associated with the trapping array as a whole. Such analysis instrumentation can comprise electrodes, a photodetector, the focal point of a microscope, or other similar sensing device. In one embodiment, the inlet and/or outlet ports comprise a sensing instrument that can measure resistance, impedance of fluid flow through the chamber, wherein a reduction in fluid flow is indicative of trapping of a biological agent.

Electrodes could be placed inside or outside of the bead pack to perform the measurements. Placing electrodes inside the pack increase the complexity of manufacturing, but has the added advantage of lowering the background resistance. Additionally, placing the electrodes inside the pack helps prevent false positives due to clogging, because the beads will filter particulates out of the pack.

Electrical resistance changes through nanocavity systems were modeled for a closely-packed array of microparticles (FIG. 9). Knowing the perimeter, and assuming saturated binding, the obstructed area of the nanocavity with binding of various size molecules can be calculated. For various size microparticles and biomolecules, the resistance increase upon binding is plotted in FIG. 9C. This analysis suggests 1 μm diameter or smaller bead sizes for ˜10% change in resistance for an average protein molecule of 50 Å in diameter. However, if a sandwich assay is conducted where a larger particle is bound to the molecule of interest after initial binding then even larger changes can be expected.

The electrical measurements through the microparticle packs or nanocavitty networks can also be applied to electrical measurements through large macroscale chromatography columns if electrodes were introduced at both ends of the column, or in another incarnation, in a centrifuge tube containing electrodes, where the analyte is driven by centrifugal force through the pack.

For impedance measurements, the aspect of avoiding double layer capacitance contributions and measuring solution resistance contributions is important. This can be done by increasing the double layer capacitance and focusing the frequency region, for example, to between 102 to 106 Hz.

In addition to changes in electrical resistance, fluidic resistance changes are expected to be much larger since it is proportional to the inverse square of the cross-sectional area as opposed to the inverse. Incorporation of a pressure transducer, either in-line with a microfluidic device or integrated into a device, could be used to detect molecular binding. Initial testing of this concept shows that fluidic resistance doubles when 7 μm biotinylated beads are subsequently coated with streptavidin.

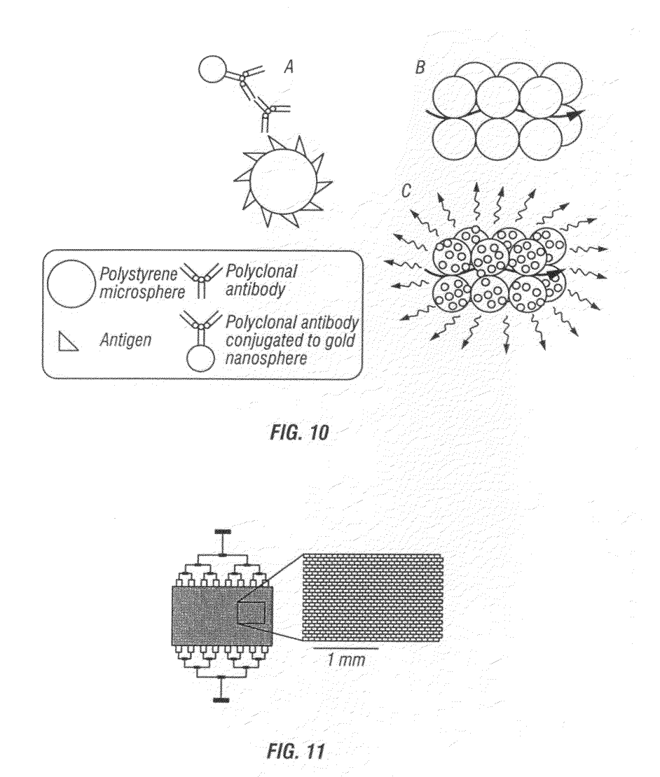

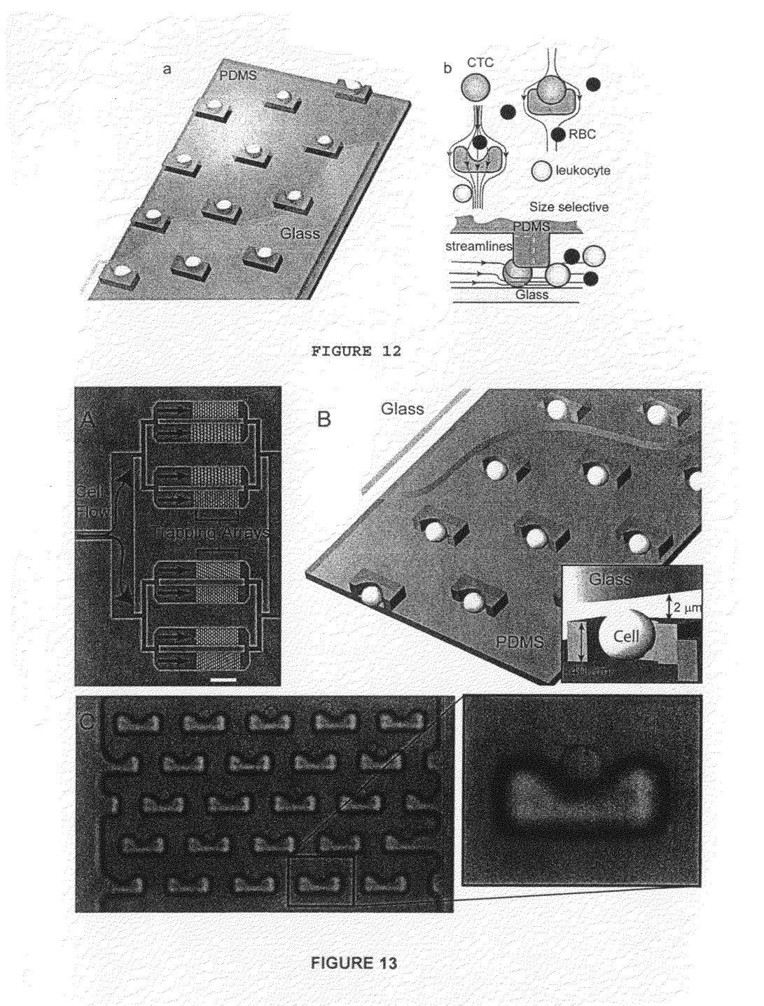

Instrument free detection. would be ideal for point-of-care diagnostic devices, and one way to achieve this is to use an immunochromatographic test. This test makes use of the fact that colloidal gold particles scatter light very efficiently. When many gold nanoparticles are grouped together, light is scattered, and a color is seen that is dependent on the nanoparticle size. Pregnancy tests use an immunochromatographic test, and have colloidal gold nanoparticles. These nanoparticles are typically sized to create a blue line. Such an assay can be performed in a microfluidic device of the invention. FIG. 10A shows the sandwich-like arrangement of the assay. Beads can be functionalized with antigen specific to antibodies the body produces in response to disease. When the sample is pushed through a micro-affinity fluidic column, the sample will travel through the void spaces between the beads (FIG. 10B). The antibody, if present, will then bind to the surfaces of the beads. Once the sample has been pushed through, a solution of gold nanoparticles attached to an anti-human antibody will be pushed through the columns. The anti-human antibody will bind to the antibodies on the surfaces of the beads if they are present. If the antibodies are not present, then the anti-human antibody will not bind to the beads, and the nanoparticles will pass through the column. When enough of the nanoparticles are captured in the column, the nanoparticles will scatter the light to an extent that is detectable with the naked eye (FIG. 10C). By combining the highly sensitive and fast micro-affinity column with a detection method that requires no additional instruments, this device can be highly successful for diagnostic screening.

This concept has been performed by binding 40 nm gold particles conjugated with streptavidin to biotinylated beads packed into a column within a microfluidic device. The bead pack turned a noticeable pink color, and was easily distinguishable from the control experiment. The control device consisted of a plain beads packed into a column. The color of the control channel remained unchanged after introduction of the colloidal gold solution because there was no specific binding between the gold and the beads.

A microfluidic flow regulator can be used in the system and methods of the invention, such as one or more of micropumps described herein, for controlling. the flow rate. For example, the pump may be a microelectromechanical (MEMS) microfluidic pump. The micropump can be operated at a predetermined frequency, which can be either substantially constant or modulated depending upon the requirements of the system.

The microfluidic device of the invention can comprise other manipulation chambers including, for example, cell lysis, cell removal, cell separation, and the like, separation of the desired target analyte from other sample components, chemical or enzymatic reactions on the target analyte, detection of the target analyte and the like. The devices of the invention can include one or more reservoirs for sample manipulation and storage, waste or reagent storage; fluid channels to and between such reservoirs, including microfluidic channels. Such channels may comprise electrophoretic separation systems (e.g., microelectrodes); valves to control fluid movement; pumps such as electroosmotic, electrohydrodynamic, or electrokinetic pumps; and detectors as more fully described herein. The devices of the invention can be designed to manipulate one or a plurality of samples or analytes simultaneously or sequentially.

As shown in FIG. 4, these components include, but are not limited to, sample inlet ports 20, outlet ports and the like. Other components can include fluid pumps; fluid valves; thermal modules for heating and cooling; storage modules for assay reagents; interaction chamber(s); and detection modules.

The at least one inlet port 20 and the at least one outlet port 40 can comprise valves to control delivery and removal of a fluid from the chamber 30.

Thus, the devices of the invention include at least one flow channel that allows the flow of sample from an inlet port 20 or reservoir to the other components or modules of the system. As will be appreciated by those in the art, the flow channels may be configured in a wide variety of ways, depending on the use of the channel. For example, a single flow channel starting at the sample inlet port may be separated into a variety of smaller channels, such that the original sample is divided into discrete subsamples for parallel processing or analysis. Alternatively, several flow channels from different modules, for example, the sample inlet port and a reagent storage module may feed together into chamber 30. As will be appreciated by those in the art, there are a large number of possible configurations; what is important is that the flow channels allow the movement of sample and reagents from one part of the device to another. For example, the path lengths of the flow channels may be altered as needed; for example, when mixing and timed reactions are required, longer flow channels can be used.

In one embodiment, the devices of the invention include at least one inlet port 20 for the introduction of the sample to the device. This may be part of or separate from the flow chamber 30 or a mixing chamber; that is, the sample may be directly fed in from the sample inlet port to a chamber comprising the plurality of weir-traps.

In another aspect of the invention, the devices of the invention may include a cell manipulation chamber. A cell manipulation chamber is useful when the sample comprises cells that either contain the target analyte or that need to be separated in to subpopulations in order to detect the target analyte or desired cell. For example, the detection of a target analyte in blood can require the removal of the blood cells for efficient analysis, or the cells (and/or nucleus) must be lysed prior to detection. In this context, “cells” include eukaryotic and prokaryotic cells, and viral particles that may require treatment prior to analysis, such as the release of nucleic acid from a viral particle prior to detection of target nucleic acids. The sample is then provided to chamber 30 comprising weir-traps 35.

In another aspect of the invention, the system comprises at least one pump. These pumps can be any type of pump device including electrode based pumps. Electromechanical pumps can be used in the systems of the invention, e.g. based upon capacitive, thermal, and piezoelectric actuation. Suitable on chip pumps include, but are not limited to, electroosmotic (EO) pumps and electrohydrodynamic (EHD) pumps; these electrode based pumps have sometimes been referred to in the art as “electrokinetic (EK) pumps”. All of these pumps rely on configurations of electrodes placed along a flow channel. As is described in the art, the configurations for each of these electrode based pumps are slightly different; for example, the effectiveness of an EHD pump depends on the spacing between the two electrodes, with the closer together they are, the smaller the voltage required to be applied to effect fluid flow. Alternatively, for EO pumps, the spacing between the electrodes should be larger, with up to one-half the length of the channel in which fluids are being moved, since the electrode are only involved in applying force, and not, as in EHD, in creating charges on which the force will act.

In one embodiment, an electroosmotic pump is used. Electroosmosis (EO) is based on the fact that the surface of many solids, including quartz, glass and others, become variously charged, negatively or positively, in the presence of ionic materials. The charged surfaces will attract oppositely charged counterions in aqueous solutions. Applying a voltage results in a migration of the counterions to the oppositely charged electrode, and moves the bulk of the fluid as well. The volume flow rate is proportional to the current, and the volume flow generated in the fluid is also proportional to the applied voltage. Electroosmotic flow is useful for liquids having some conductivity and generally not applicable for non-polar solvents.

In another embodiment, an electrohydrodynamic (EHD) pump is used. In EHD, electrodes in contact with the fluid transfer charge when a voltage is applied. This charge transfer occurs either by transfer or removal of an electron to or from the fluid, such that liquid flow occurs in the direction from the charging electrode to the oppositely charged electrode. EHD pumps can be used to pump resistive fluids such as non-polar solvents.

In another aspect, the pumps are external to the microfluidic device or chamber 30. In this aspect, the pump may be a peristaltic pump, syringe pump or other pump commonly used in the art.

In another aspect of the invention, the devices of the invention include at least one fluid valve that can control the flow of fluid into or out of a module or chamber of the device or divert the flow into one or more channels. A variety of valves are known in the art. For example, in one embodiment, the valve may comprise a capillary barrier, as generally described in PCT US97/07880, incorporated by reference. In this embodiment, the channel opens into a larger space designed to favor the formation of an energy minimizing liquid surface such as a meniscus at the opening. Typically, capillary barriers include a dam that raises the vertical height of the channel immediately before the opening into a larger space such a chamber. In addition, as described in U.S. Pat. No. 5,858,195, incorporated herein by reference, a type of “virtual valves” can be used.

In yet another embodiment, the devices of the invention include sealing ports, to allow the introduction of fluids, including samples, into any of the modules of the invention, with subsequent closure of the port to avoid the loss of the sample.

The devices of the invention can include at least one storage modules for assay reagents (e.g., buffer, sample, binding agent). These are connected to other modules of the system using flow channels and may comprise wells or chambers, or extended flow channels. They may contain any number of reagents, buffers, enzymes, electronic mediators, salts, and the like, including freeze dried reagents.

In another embodiment, the devices of the invention include a mixing module; again, as for storage modules, these may be extended flow channels (particularly useful for mixing), wells or chambers. Particularly in the case of extended flow channels, there may be protrusions on the side of the channel to cause mixing.

The devices of the invention can include a detection module. For example, the detection module can incorporate both electrical sensing and optical illumination to enable a scheme where the label probes or cells include multiple detection moieties that are photochemically dissociated to amplify the detected signal from a single probe above the background threshold.

In one aspect, the detection modules of the invention comprise electrodes. By “electrode” herein is meant a composition, which, when connected to an electronic device, is able to sense a current or charge and convert it to a signal. Alternatively an electrode can be defined as a composition which can apply a potential to and/or pass electrons to or from species in the solution. Electrodes are known in the art and include, but are not limited to, certain metals and their oxides, including gold; platinum; palladium; silicon; aluminum; metal oxide electrodes including platinum oxide, titanium oxide, tin oxide, indium tin oxide, palladium oxide, silicon oxide, aluminum oxide, molybdenum oxide (Mo₂O₆), tungsten oxide (WO₃) and ruthenium oxides; and carbon (including glassy carbon electrodes, graphite and carbon paste).

In another aspect, the detector can be an optical detector capable of detecting an optical change. The change in optics may be the result of the presence of a luminescence or fluorescence label associated with an aggregate or cell.

In one embodiment, electronic detection is used, including amperommetry, voltammetry, capacitance, and impedance. Suitable techniques include, but are not limited to, electrogravimetry; coulometry (including controlled potential coulometry and constant current coulometry); voltametry (cyclic voltametry, pulse voltametry (normal pulse voltametry, square wave voltametry, differential pulse voltametry, Osteryoung square wave voltametry, and coulostatic pulse techniques); stripping analysis (aniodic stripping analysis, cathiodic stripping analysis, square wave stripping voltammetry); conductance measurements (electrolytic conductance, direct analysis); time-dependent electrochemical analyses (chronoamperometry, chronopotentiometry, cyclic chronopotentiometry and amperometry, AC polography, chronogalvametry, and chronocoulometry); AC impedance measurement; capacitance measurement; AC voltametry; and photoelectrochemistry.

Accordingly, the invention provides a device for the detection of target analytes or biological agents (including cells) comprising a substrate with a plurality of weir-traps. The substrate can be made of a wide variety of materials and can be configured in a variety of designs. In some cases, a portion of the substrate may be removable; for example, the substrate/cover defining chamber 30 may be a detachable cassette that can be removed from the device following use.

The devices of the invention can be made in a variety of ways, as will be appreciated by those in the art. Suitable fabrication techniques again will depend on the choice of substrate. Exemplary methods include, but are not limited to, a variety of micromachining and microfabrication techniques, including film deposition processes such as spin coating, chemical vapor deposition, laser fabrication, photolithographic and other etching techniques using either wet chemical processes or plasma processes, embossing, injection molding and bonding techniques. In addition, there are printing techniques for the creation of desired fluid guiding pathways; that is patterns of printed material can permit directional fluid transport.

In addition, it should be understood that while most of the discussion herein is directed to the use of planar substrates with weir-traps, other geometries can be used as well. For example, two or more planar substrates can be stacked to produce a three dimensional device, that can contain weir-traps flowing within one plane or-between planes.

In methods and systems of the invention microfluidic handling of microparticles assists in both the formation of aggregates and the detection of the aggregation event. These techniques allow for inexpensive, label-free, easily operated biomolecular detection methods for diagnostic applications (immunosensing and DNA hybridization detection). Detection can use a simple electrical, pressure, and naked-eye optical method based on the accumulation of aggregates in a microfluidic chamber. To assist in aggregation, microfluidic methods are used to alternately coat layers of recognition element-bound-microparticles and the detected biomolecule, overcoming the non-linear concentration dependence difficulties in aggregation assays (FIG. 7).

The method utilizes a device as described herein comprising: (1) a semipermeable structure to hold microparticles/nanoparticles (e.g., a weir-trap); (2) switched exposure between molecule to bind to the particle and additional particles to bind to the coated particles; and (3) molecules and recognition elements that bind at more than one location (e.g. polyclonal antibodies). Additional methods and features can comprise: (1) a method to amplify the continued aggregation events, such as complete impermeability of the channel to additional particles—This leads to build up of particles in a microchannel and a naked-eye visible aggregate that will indicate molecule presence; (2) large amounts of beads occupying a microchannel can also be measured electrically, using electrodes on chip and high frequency impedance measurements in the range 10² to 10⁶ Hz to measure solution resistance dominated region as opposed to double layer capacitance dominated region. Electrochemical DC and AC measurements can be used where charge transfer is occurring across the electrode.

Examples of binding ligands (e.g., biological molecules) that can be used in the methods and systems of the invention include molecules (e.g., polymers) typically found in living organisms. Examples include, but are not limited to, proteins, nucleic acids, lipids, and carbohydrates.

As used herein, the term “target analyte” and “biological agent” refer to a molecule or organism in a sample to be detected. Examples of target analytes include, but are not limited to, polynucleotides, oligonucleotides, viruses, polypeptides, antibodies, naturally occurring drugs, synthetic drugs, pollutants, allergens, affector molecules, growth factors, chemokines, cytokines, and lymphokines.

Biological agents include organic and inorganic molecules, including biological molecules. For example, the analyte may be an environmental pollutant (including pesticides, insecticides, toxins, and the like); a chemical (including solvents, polymers, organic materials, and the like); therapeutic molecules (including therapeutic and abused drugs, antibiotics, and the like); biological molecules (including, e.g., hormones, cytokines, proteins, lipids, carbohydrates, cellular membrane antigens and receptors (neural, hormonal, nutrient, and cell surface receptors) or their ligands); whole cells (including prokaryotic and eukaryotic cells; viruses (including, e.g., retroviruses, herpesviruses, adenoviruses, lentiviruses); spores; and the like. Additional examples of target analytes and biological agents include, but are not limited to, immunoglobulins, particularly IgEs, IgGs and IgMs, and particularly therapeutically or diagnostically relevant antibodies, including but not limited to, antibodies to human albumin, apolipoproteins (including apolipoprotein E), human chorionic gonadotropin, cortisol, α-fetoprotein, thyroxin, thyroid stimulating hormone (TSH), antithrombin, antibodies to pharmaceuticals (including antieptileptic drugs (phenytoin, primidone, carbariezepin, ethosuximide, valproic acid, and phenobarbitol), cardioactive drugs (digoxin, lidocaine, procainamide, and disopyramide), bronchodilators (theophylline), antibiotics (chloramphenicol, sulfonamides), antidepressants, immunosuppresants, abused drugs (amphetamine, methamphetamine, cannabinoids, cocaine and opiates) and antibodies to any number of viruses (including orthomyxoviruses, (e.g., influenza virus), paramyxoviruses (e.g., respiratory syncytial virus, mumps virus, measles virus), adenoviruses, rhinoviruses, coronaviruses, reoviruses, togaviruses (e.g., rubella virus), parvoviruses, poxviruses (e.g., variola virus, vaccinia virus), enteroviruses (e.g., poliovirus, coxsackievirus), hepatitis viruses (including A, B and C), herpesviruses (e.g., Herpes simplex virus, varicella-zoster virus, cytomegalovirus, Epstein-Barr virus), rotaviruses, Norwalk viruses, hantavirus, arenavirus, rhabdovirus (e.g., rabies virus), retroviruses (including HIV, HTLV-I and -II), papovaviruses (e.g., papillomavinus), polyomaviruses, and picornaviruses, and the like), and bacteria (including a wide variety of pathogenic and non-pathogenic prokaryotes of interest including Bacillus; Vibrio, e.g., V. cholerae; Escherichia, e.g., Enterotoxigenic E. coli, Shigella, e.g., S. dysenteriae; Salmonella, e.g., S. typhi; Mycobacterium e.g., M. tuberculosis, M. leprea; Clostridium, e.g., C. botulinum, C. teteni, C. difficile, C. perfringens; Cornyebacterium, e.g., C. diphtheriae; Streptococcus, S. pyogenes, S. pneumoniae; Staphylococcus, e.g., S. aureus; Haemophilus, e.g., H. influenzae; Neisseria, e.g., N. meningitidis, N. gonorrhoeae; Yersinia, e.g., G. lamblia, Y. pestis, Pseudomonas, e.g., P. aeruginosa, P. putida; Chlamydia, e.g., C. trachomatis; Bordetella, e.g., B. pertussis; Treponema, e.g., T. palladium; and the like); enzymes (and other proteins), including, but not limited to, enzymes used as indicators of or treatment for heart disease, including creatine kinase, lactate dehydrogenase, aspartate amino transferase, troponin T, myoglobin, fibrinogen, cholesterol, triglycerides, thrombin, tissue plasminogen activator (tPA); pancreatic disease indicators including amylase, lipase, chymotrypsin and trypsin; liver function enzymes and proteins including cholinesterase, bilirubin, and alkaline phosphatase; aldolase, prostatic acid phosphatase, terminal deoxynucleotidyl transferase, and bacterial and viral enzymes such as HIV protease; hormones and cytokines (many of which serve as ligands for cellular receptors) such as erythropoietin (EPO), thrombopoietin (TPO), the interleukins (including IL-1 through IL-17), insulin, insulin-like growth factors (including IGF-1 and -2), epidermal growth factor (EGF), transforming growth factors (including TGF-α and TGF-β), human growth hormone, transferrin, epidermal growth factor (EGF), low density lipoprotein, high density lipoprotein, leptin, VEGF, PDGF, ciliary neurotrophic factor, prolactin, adrenocorticotropic hormone (ACTH), calcitonin, human chorionic gonadotropin, cortisol, estradiol, follicle stimulating hormone (FSH), thyroid-stimulating hormone (TSH), leutinzing hormone (LH), progeterone and testosterone; and other proteins (including α-fetoprotein, carcinoembryonic antigen CEA, cancer markers, and the like). In addition, any of the biomolecules that are indirectly detected through the use of-antibodies may be detected directly as well; that is, detection of virus or bacterial cells, therapeutic and abused drugs, and the like, may be done directly.

Suitable target analytes include carbohydrates, including, but not limited to, markers for breast cancer (CA15-3, CA 549, CA 27.29), mucin-like carcinoma associated antigen (MCA), ovarian cancer (CA125), pancreatic cancer (DE-PAN-2), prostate cancer (PSA), CEA, and colorectal and pancreatic cancer (CA 19, CA 50, CA242). Suitable target analytes also include metal ions, particularly heavy and/or toxic metals, including but not limited to, aluminum, arsenic, cadmium, selenium, cobalt, copper, chromium, lead, silver and nickel.

Target analytes and biological agents may be present in any number of different sample types, including, but not limited to, bodily fluids including blood, lymph, saliva, vaginal and anal secretions, urine, feces, perspiration and tears, and solid tissues, including liver, spleen, bone marrow, lung, muscle, brain, and the like. For example, in its broadest sense a sample includes, but is not limited to, environmental, industrial, and biological samples. Environmental samples include material from the environment such as soil and water. Industrial samples include products or waste generated during a manufacturing process. Biological samples may be animal, including, human, fluid (e.g., blood, plasma and serum), solid (e.g., stool), tissue, liquid foods (e.g., milk), and solid foods (e.g., vegetables).

Binding ligands, partners or cognates refers to two molecules (e.g., proteins) that are capable of, or suspected of being capable of, physically interacting with each other. Two nucleic acid molecules capable of hybridizing to one another due to complementarity are to be understood as binding partners where the context is appropriate. Aggregates can be formed through the interactions of binding ligands and their associated binding partner.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

The working examples below are provided to illustrate, not limit, the invention. Various parameters of the scientific methods employed in these examples are described in detail below and provide guidance for practicing the invention in general.

### EXAMPLES

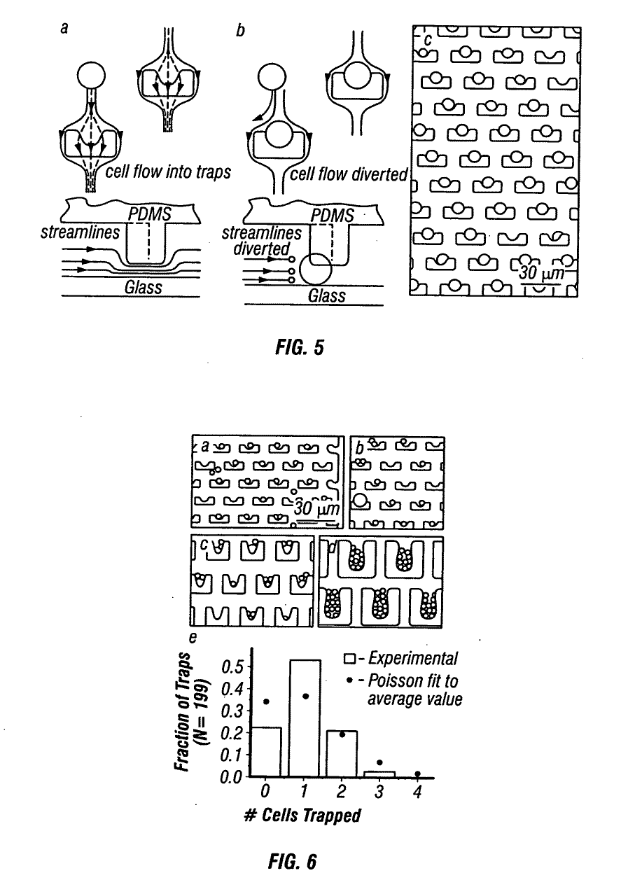

During use of one aspect of the invention, cells were introduced through a branching inlet port of a microfluidic device to individual chambers comprising trapping arrays (FIG. 4). Single cells were isolated in regular high density arrays composed of two channel height levels. A larger 40 μm channel height serves as the main fluid conduits for cell solutions, while the 2 μm height regions was used to form elevated trapping regions (FIG. 5). Having a 2 μm gap allowed a fraction of fluid streamlines carrying cells to enter a trap. Once a cell enters a trap and partially occluded the 2 μm gap the fraction of fluid streamlines (and cells) entering the weir-trap trap region were reduced. This leads to a high quantity of single cell isolates (FIG. 5, FIG. 6a-e). In effect the probability of trapping was dependent on the number of cells previously trapped. This is shown statistically, by comparing a Poisson distribution to the experimentally measured distribution in FIG. 6e (N=199 trapping sites, 4 separate loadings). If trapping were independent of the previous trapping events the data should follow a Poisson distribution. Here single cells were shown in excess of the Poisson distribution while zero, three, and four trapped cells are depressed. If one tries to explain trapping to be purely dependent on geometrical fit then trapping of two cells is expected to be more common since the channel height is more than double the diameter of the typical mammalian cell (40 μm compared to 15 μm).

As discussed herein, various weir-trap sizes and geometries can be used. Maintaining the same weir-trap width and channel height, the depth of the weir-traps can be varied from 10 to 60 μm. The depth of the trapping structures, signifying the “deepness” of the pocket, should not be confused with the channel depth which is referred to as “height”. These various depths resulted in differences in the number of trapped cells (FIG. 6). For the 10 μm deep weir-traps>50% of weir-traps contained single cells, with a decreasing fraction of single cell isolates as weir-trap depth increased. The distribution of number of cells trapped for the 10 μm deep weir-traps is shown in FIG. 6e. The density of the array also effects trapping efficiency of single cells since, excess cells experience a higher shear force and are removed from less stable positions.

Devices were found to be quite effective and easy to use, with trapping able to be conducted in less than 30 seconds. Also, demonstrating the robustness of the method, the device has been successfully fabricated and operated. Differences in this method when comparing to other fluid mechanical methods of trapping include “self-sealing” of the trap as the resistance increases, geometrical tuning to enhance single cell isolates with a simple flow through procedure, and high density arrayability. To compare with flow cytometry (FC), although FC can interrogate hundreds of thousands of cells, and at several time points, analysis of the same cell over a long period of time is not possible, nor is positional dependent analysis of fluorescence within individual cells. Particularly difficult to probe with FC is fast time dependent changes upon addition of a compound to a cell. The device and techniques presented have the potential to bridge all of these gaps in FC, with moderate throughput.

Dynamic cellular analysis has been demonstrated by observing cell growth and division as well as enzyme content in arrays of individual cells. Cells can be maintained for long periods of time in culture with correct media, and especially useful is culture of suspension cells in a fixed location, that do not attach to surfaces and are difficult to observe using other methods.

Microfluidic chip fabrication. The molds for the trapping array culture device were fabricated using negative photoresists (SU-8 50 and SU-8 2002, Microchem Corporation, 3000 rpm spin speed, 40 μm and 2 μm thick) as in Di Carlo et al. Poly-dimethylsiloxane (PDMS, Sylgard 184, Dow Corning) was prepared according to the manufacturers instructions, degassed in a vacuum chamber for 1 hour and then poured on the mold and cured in a 70° C. oven for 2 hours. The PDMS was cut from the mold with a surgical scalpel and then carefully peeled off the mold. The fluid inlet and outlet were punched by a flat-tip needle for tube connections. Both a glass slide and the PDMS structures were treated with oxygen plasma (0.5 torr, 40 W) for 20 seconds before bonding.

Cell culture and preparation. HeLa (human cervical carcinoma) cell line was used in experiments (American Type Culture Collection, Bethesda, Md.). The cells were maintained by passaging twice weekly with Dubelcco's Modified Eagle Medium (DMEM) supplemented with 10% fetal bovine serum (FBS). For loading, adherent cells were detached from 100 mm diameter culture dishes with 5 mL Trypsin EDTA (0.25%, Gibco, Carlsbad, Calif.). An equal amount of DMEM+FBS was then added to deactivate remaining Trypsin. Cells were then centrifuged to a pellet and resuspended in phosphate buffered saline pH 7.4 (PBS, Gibco, Carlsbad, Calif.). A key experimental detail is to trap suspended cells within 15 minutes of trypsinization to reduced non-specific adhesion to surfaces. Freshly suspended cells were introduced into previously PBS filled devices by a syringe connected to a three way valve. For control experiments, suspended cells were introduced onto a glass slide contained by a PDMS well and cultured in either an incubator or (37° C.) heated stage for time-lapse experiments.

On-chip cell culture. Tubing, valves and devices were first sterilized with 70% ethanol for 5 minutes prior to loading. Sterile PBS was then used to prime the device and tubing. The previous steps were all done within a biosafety hood to reduce contamination. Then, cell solution was added and cells trapped to the desired density. Next, a valve was switched to sterile media +10% FBS and flow was initiated to perfuse the cells. A flow rate of 0.75 μl min⁻¹ using a syringe pump (Cole-Parmer) yielded an average velocity of ˜25 μm s⁻¹ in the trapping region. Cells were either maintained in an incubator between images, or were heated to 37° C. on a microscope stage for time-lapse experiments.

Microscopy and Data Analysis. For time-lapse experiments an Olympus MIC-D microscope was outfitted with a heated stage. Time-lapse images were collected using the provided MIC-D software every 3 to 6 minutes. Images were analyzed to determine morphology and cell division using IrfanView. Cells with a long axis 1.3 times the short axis were considered to be “adherent”. Cells were identified as dividing if they retracted from adherent morphology, became spherical and then separated into two daughter cells. Cells were identified as apoptotic if they showed blobbing, no movement or shape change over 6 hours, or other apoptotic characteristics.

Device Modeling. For cell culture it is important to understand and control the shear stress on cell surfaces, since cell pathways can be activated by high shear stress leading to unwanted cell behavior. The flow fields around an isolated trapping structure were modeled in 3D using the finite element method (FEMLAB 3.0, Comsol Inc.). The Navier-Stokes equations were used to model fluid flow with only viscous terms (i.e. ρ=0 in the subdomains). Boundary conditions consisted of an average velocity of 25 μm s⁻¹ at the inlet and pressure set to 0 at the outlet. The side walls of the computational domain were set to symmetry, simulating a row of trapping structures. A single trap (coarse mesh) was simulated instead of an array. Both velocities throughout the domain and shear stress components at the boundaries of the domain were collected.

Trapping arrays were successfully fabricated and tested. The device consists of branched trapping chambers linked in parallel (FIG. 13A), while the arrays within the chambers consist of U-shaped PDMS structures that are 40 μm in height and are offset from the substrate by 2 μm (FIG. 13B-C). Each chamber contained between 4 and 5 traps over its width (FIG. 13C). Also, each row of traps was asymmetrically offset from the previous row (FIG. 13C). It was qualitatively observed that asymmetric rows of traps were better at filling throughout the chamber when compared to symmetrically offset rows. Several lengths for the depth of the trap were examined for the best isolation of individual cells (10 μm, 15 μm, 30 μm, and 60 μm). It was found that ten micrometer deep traps most consistently trapped individual HeLa cells (average diameter ˜15 μm). For other cell types with different average diameters, the optimum trap size should vary. Additionally, since there is a distribution of cell sizes amongst a population, there may be a bias to trap smaller cells that can more easily occupy the trapping sites.

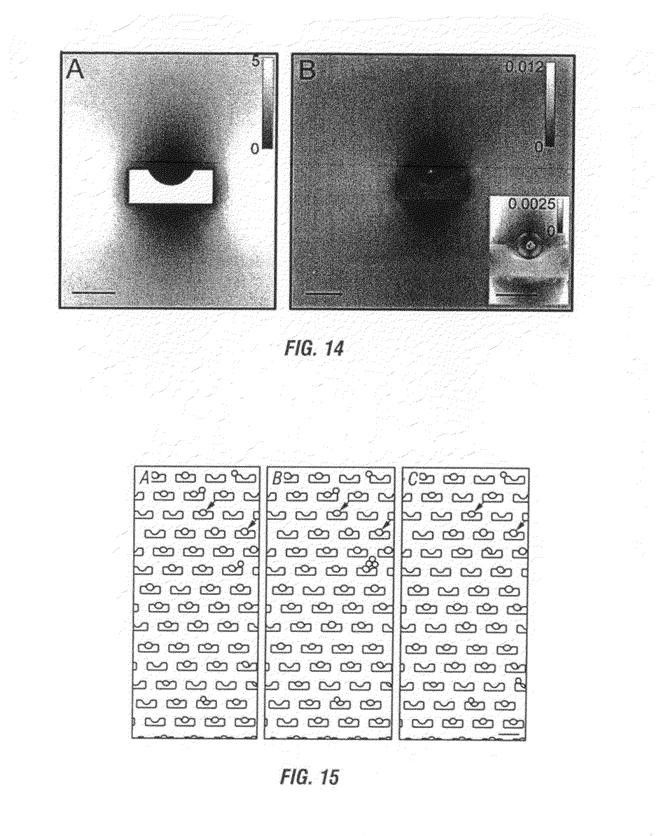

The fluid velocity and shear stress were simulated as described in Materials in Methods for a single 3D trap structure containing a spherical trapped cell. This was conducted to determine shear stress conditions for trapped cells, to compare to physiologically relevant shear stresses. For a flow rate of 0.75 μL min⁻¹ used in experiments the maximum velocity reaches ˜50 μm s⁻¹ and the distribution of velocity magnitudes around a single occupied trap is shown in FIG. 14A at a position z=20 μm from the substrate in the middle of the channel. In the region in front of and behind the trap the velocity is reduced as is expected. Shear stresses on a spherical trapped cell were also modeled and the distribution is plotted over the trapped cell and on the bottom surface of the channel for the same flow conditions (FIG. 14B). The shear stress of the bottom surface approximates that which an adherent cell would feel. Here, the average shear stress, observed outside the trapping structure is 6×10⁻² dyn cm⁻² and the average shear stress in the trap is 2.5×10⁻³ dyn cm⁻². This leads to a ratio of shear stress between the main flow and within the trap of ˜24. The average shear stress on a spherical trapped cell is also 3.5×10⁻³ dyn cm⁻². These numbers are much below physiological shear stress of ˜10 dyn cm⁻² that vascular endothelial cells experience but comparable to shear stress caused by interstitial flow. The shear stress ratio observed in the device will remain independent of flow rate for low Reynold's number and is a number characterizing how “shielded” the trapped single cells will be from the main flow.

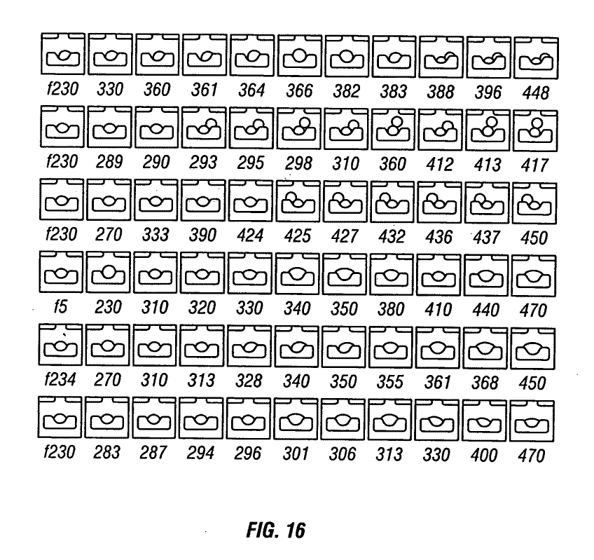

Cultures of ordered arrays of single HeLa cells under constant perfusion of media +10% FBS-were obtained. For a flow rate of 25 μL min 3 time-lapse images were taken every 3 minutes of a trapped array of HeLa cells on an incubated microscope stage (FIG. 15). Cells are shown after 12 and 24 hours in FIG. 15B-C. Initially, the single cell trapping rate for this sequence was 70% (FIG. 15A). After 12 hours small changes in morphology are observed away from a spherical morphology towards an adherent morphology. Also, cell division is observed in a few cases (top red arrow - FIG. 15B). After 24 hours, a majority of cells display an adherent morphology and both cells identified with arrows have divided. In some cases cells are observed to escape the trapping structures as well. Behavior of several cells in the trapping structure over time is shown for dividing and adhering cells in FIG. 16. It should be noted that in most cases after cell division both daughter cells remain isolated in the trapping structure. Another interesting observation is the directionality of adherence in HeLa cells that are trapped. It is observed that a large fraction of growing HeLa cells have a long axis parallel to the long axis of the trapping structure. It also appears that the cells became adherent to the PDMS structure as opposed to the glass substrate in these cases. This may be due to serum containing adhesion-promoting proteins that may adhere to the hydrophobic PDMS surface biasing attachment. Adhesion on the PDMS structures may limit microscopic analysis in some cases, due to diffraction at the interface of the trap. To limit adhesion, future studies could employ treatments with high concentrations of bovine serum albumin (BSA) that will coat the PDMS surface.

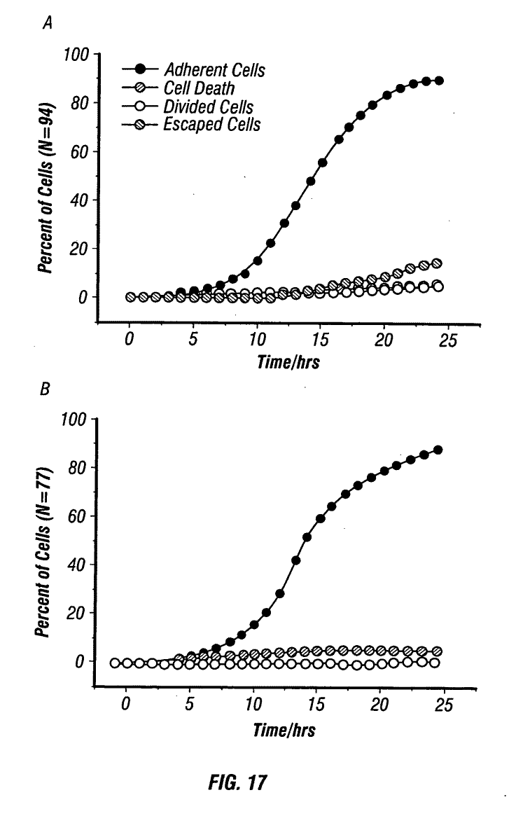

Quantitative analysis of the dynamics of cell adhesion, death, division, and escape from traps were performed for a 24 hour period and are plotted in FIG. 17A. Here it was observed that 50% of cells displayed adherent morphology after 15 hours. After 24 hours 6% of cells showed characteristics of apoptosis, while 15% had escaped from the vicinity of the initial trapping site. The high level of maintenance within the trapping structures after 24 hours may be due shear sheltering within the trapping structure. Additionally, 5% of cells had undergone cell division after 24 hours. These results were compared to cell behavior in a control experiment using the same glass substrate with no traps or perfusion (FIG. 17B). In this experiment 50% of cells were adherent after a similar 14 hours, while 5% of cells were apoptotic after 24 hours, and only 1% of cells had undergone cell division. The requirement for a cell to be considered “adherent” was a length 1.3× its width.

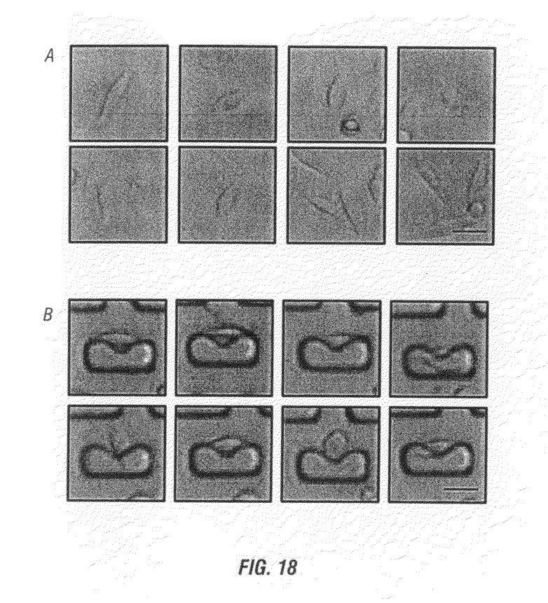

Adherent morphology was confirmed by comparing cell behavior in the trapping structure to cells cultured under similar conditions in the control experiment (FIG. 18). Similar adherent and elongated morphology is observed in the images seen on a glass slide (FIG. 18A) and in the device (FIG. 18B).

The invention provides a microfluidic-based hydrodynamic trapping method for creating arrays of single adherent cells with dynamic control of perfusion possible. HeLa cells are cultured and a high level of maintenance in the original position of trapping is observed after 24 hours. Additionally, cell division, adhesion, and apoptotic behavior was comparable to static culture on the same substrate, indicating cells are not stressed above normal culture conditions. After cell division, daughter cells were also observed to be maintained within the original trapping structure. As compared with previous single cell arrays, cell-cell communication by both contact and diffusible elements is a controllable parameter in this device. This technique will be useful in single cell studies of metabolism, pharmacokinetics, drug toxicity, shear stress activation, and chemical signaling pathway activation and inhibition.

1. A microfluidic device comprising:
an inlet;
an outlet; and
at least one substrate, wherein the substrate is disposed between and in fluid communication with the inlet and outlet port;
a cover, wherein the cover and the at least one substrate define an internal flow chamber, the flow chamber having a height;
a plurality of weir-traps, wherein the plurality of weir-traps are disposed on the substrate and extend into the internal flow chamber, the plurality of weir-traps having a height less than the height of the flow chamber; and
at least one detection device, wherein the at least one detection device measures flow dynamics or electrical properties through the chamber.

2. The microfluidic device of claim 1, further comprising a buffer reservoir fluidly connected to the chamber.

3. The microfluidic device of claim 1, further comprising a means for measuring fluid flow through the chamber.

4. The microfluidic device of claim 1, further comprising an electrode for electrically measuring an analyte or biological agent located with the chamber.

5. The microfluidic device of claim 1, further comprising a plurality of beads located between at least two weir-traps of the plurality of weir-traps within the chamber.

6. The microfluidic device of claim 5, wherein the plurality of beads are functionalized to bind an analyte or biological agent.

7. The microfluidic device of claim 5, wherein the beads comprise different diameters.

8. The microfluidic device of claim 5, wherein the beads comprise bound nanoparticles.

9. The microfluidic device of claim 5, wherein the beads are functionalized with a nucleic acid and/or a polypeptide.

10. The microfluidic device of claim 1, comprising a means for inducing fluid flow through the chamber.

11. The microfluidic device of claim 8, wherein said means for inducing flow comprises at least one pump.

12. The microfluidic device of claim 1, further comprising a plurality of valves operational to begin, stop or reduce fluid flow through the system.

13. The microfluidic device of claim 1, wherein the cover is movable within the chamber.

14. The microfluidic device of claim 13, further comprising means for measuring changes in electrical resistance through the beads.

15. The microfluidic device of claim 1, further comprising means for measuring analytes or biological agents associated with a weir trap by measuring changes in fluidic resistance or pressure within the chamber.

16. The microfluidic device of claim 5, further comprising means for measuring analytes or biological agents associated with a bead by measuring changes in fluidic resistance or pressure within the chamber.

17. The microfluidic device of claim 1, wherein the plurality of weir-traps are designed to trap an analyte and promote agglutination.

18. The microfluidic device of claim 1, wherein the plurality of weir-traps are designed to trap a biological agent.

19. The microfluidic device of claim 18, wherein the biological agent is a cell or viral particle.

20. A method for the detection of a target analyte or biological agent in a fluid sample comprising:
contacting the sample with a microfluidic device of claim 1 and detecting a change selected from the group consisting of resistance, flow, fluid pressure, and optics.
